(19) United States
(12) Patent Application Publication
(10) Pub. No.: US 2016/0302713 A1
(43) Pub. Date: Oct. 20, 2016

(54) System and Method for Concussion Detection and Quantification

(71) Applicant: Sync-Think, Inc., Boston, MA (US)

(72) Inventors: Jun Maruta, New York, NY (US); Umesh Rajashekar, New York, NY (US); Jamshid Ghajar, Palo Alto, CA (US)

(21) Appl. No.: 15/099,427

(22) Filed: Apr. 14, 2016

Related U.S. Application Data
(60) Provisional application No. 62/148,094, filed on Apr. 15, 2015.

(51) Int. Cl. A61B 5/00; A61B 3/14; A61B 5/0484; A61B 3/113; A61B 3/00

(52) U.S. Cl. CPC A61B 5/4088; A61B 3/113; A61B 3/0091; A61B 3/0025; A61B 5/7282; A61B 5/04842; A61B 3/0041; A61B 3/145; A61B 5/6803; A61B 5/04845

(57) ABSTRACT

A method of testing a subject for impairment includes presenting the subject with a display of a moving object, repeatedly moving over a tracking path and, while presenting the display to the subject, measuring the subject's measuring the subject's gaze positions. A computer system generates tracking error data corresponding to differences in the measured gaze positions and corresponding positions of the moving object, filters the tracking error data to remove data meeting one or more predefined thresholds so as to generate filtered tracking error data, and generates a representation (e.g., a visual representation) of the filtered tracking error data, the representation indicating frequency and amplitude of anticipatory saccades in subject's visual tracking of the moving object.

### RELATED APPLICATIONS

This application claims priority to U.S. Provisional Patent Application No. 62/148,094, filed Apr. 15, 2015, entitled “System and Method for Concussion Detection and Quantification”, which is incorporated herein by reference in its entirety.

This application is related to U.S. application Ser. No. 14/454,662, filed Aug. 7, 2014, entitled “System and Method for Cognition and Oculomotor Impairment Diagnosis Using Binocular Coordination Analysis,” which is incorporated herein by reference in its entirety.

This application is also related to U.S. application Ser. No. 11/245,305, filed Oct. 5, 2005, entitled “Cognition and Motor Timing Diagnosis Using Smooth Eye Pursuit Analysis,” now U.S. Pat. No. 7,819,818, which is incorporated herein by reference in its entirety.

### TECHNICAL FIELD

The disclosed embodiments relate generally to systems and methods of testing a person's ability to track and anticipate visual stimuli, and more specifically, to a method and system for detecting and generating metrics corresponding to anticipatory saccades in a person's visual tracking of a smoothly moving object, the presence of which has been found to be indicative of concussion or other neurological, psychiatric or behavioral condition.

### BACKGROUND

Pairing an action with anticipation of a sensory event is a form of attention that is crucial for an organism's interaction with the external world. The accurate pairing of sensation and action is dependent on timing and is called sensory-motor timing, one aspect of which is anticipatory timing. Anticipatory timing is essential to successful everyday living, not only for actions but also for thinking. Thinking or cognition can be viewed as an abstract motor function and therefore also needs accurate sensory-cognitive timing. Sensory-motor timing is the timing related to the sensory and motor coordination of an organism when interacting with the external world. Anticipatory timing is usually a component of sensory-motor timing and is literally the ability to predict sensory information before the initiating stimulus.

Anticipatory timing is essential for reducing reaction times and improving both movement and thought performance. Anticipatory timing only applies to predictable sensory-motor or sensory-thought timed coupling. The sensory modality (i.e., visual, auditory etc.), the location, and the time interval between stimuli, must all be predictable (i.e., constant, or consistent with a predictable pattern) to enable anticipatory movement or thought.

Without reasonably accurate anticipatory timing, a person cannot catch a ball, know when to step out of the way of a moving object (e.g., negotiate a swinging door), get on an escalator, comprehend speech, concentrate on mental tasks or handle any of a large number of everyday tasks and challenges. This capacity for anticipatory timing can become impaired with sleep deprivation, aging, alcohol, drugs, hypoxia, infection, clinical neurological conditions including but not limited to Attention Deficit Hyperactivity Disorder (ADHD), schizophrenia, autism and brain trauma (e.g., a concussion). For example, brain trauma may significantly impact a person's cognition timing, one aspect of which is anticipatory timing. Sometimes, a person may appear to physically recover quickly from brain trauma, but have significant problems with concentration and/or memory, as well as having headaches, being irritable, and/or having other symptoms as a result of impaired anticipatory timing. In addition, impaired anticipatory timing may cause the person to suffer further injuries by not having the timing capabilities to avoid accidents.

### SUMMARY

Accordingly, there is a need to test a subject's sensory-motor timing and especially a subject's anticipatory timing. In accordance with some embodiments, a method, system, and computer-readable storage medium are proposed for detecting cognitive impairment, and in particular detecting cognitive impairment corresponding to concussion or other traumatic brain injury, through the analysis of tracking error data corresponding to differences between a subject's measured gaze positions and corresponding positions of a moving object that the subject is attempting to visually track. In some embodiments, a computer system generates tracking error data corresponding to differences in the measured gaze positions and corresponding positions of the moving object, filters the tracking error data to remove data meeting one or more predefined thresholds so as to generate filtered tracking error data, and generates a representation (e.g., a visual representation) of the filtered tracking error data, the representation indicating frequency and amplitude of anticipatory saccades in subject's visual tracking of the moving object.

### BRIEF DESCRIPTION OF THE DRAWINGS

FIG. 1 is a block diagram illustrating a system for measuring a subject's ability to visually track a smoothly moving object in accordance with some embodiments.

FIG. 2 is a conceptual block diagram illustrating a cognition timing diagnosis and training system in accordance with some embodiments.

FIG. 3 is a detailed block diagram illustrating a cognition timing diagnosis and training system in accordance with some embodiments.

FIGS. 4A-4F illustrate a smoothly moving object, moving over a tracking path in accordance with some embodiments.

FIG. 5A depicts gaze positions of a patient with traumatic brain injury while visually tracking a smoothly moving object following a circular path.

FIG. 5B depicts the gaze positions shown in FIG. 6A, plotted with reference to the position of the object, showing visual tracking errors having both radial and tangential components.

FIG. 5C depicts the same data shown in FIG. 5B, but excluding data having tracking errors with a magnitude, with respect to radial and/or tangential components of the tracking errors, that is less than a first threshold.

FIG. 5D depicts the same data shown in FIG. 5B, but excluding data having tracking errors with a positive phase error (tangential tracking path error) less than a second threshold.

FIGS. 5E, 5F and 5G are three examples of weighting functions used to weight gaze positions in accordance with their phase errors.

Like reference numerals refer to corresponding parts throughout the several views of the drawings.

### DETAILED DESCRIPTION OF EMBODIMENTS

While physical movement by a subject can be measured directly, cognition, which is thinking performance, must be inferred. However, since cognition and motor timing are linked through overlapping neural networks, diagnosis and therapy can be performed for anticipatory timing difficulties in the motor and cognitive domains using motor reaction times and accuracy. In particular, both the timing and accuracy of a subject's movements can be measured. As discussed below, these measurements can be used for both diagnosis and therapeutic indications.

Anticipatory cognition and movement timing are controlled by essentially the same brain circuits. Variability or a deficit in anticipatory timing produces imprecise movements and is indicative of disrupted thinking, such as difficulty in concentration, memory recall, and carrying out both basic and complex cognitive tasks. Such variability and/or deficits leads to longer periods of time to successfully complete tasks and also leads to more inaccuracy in the performance of such tasks. Accordingly, in some embodiments, such variability is measured to determine whether a person suffers impaired anticipatory timing. In some embodiments, a sequence of stimuli is used in combination with a feedback mechanism to train a person to improve anticipatory timing.

As discussed in more detail below, in some embodiments, sequenced stimuli presented to a subject are or include predictable stimuli, for example, a smoothly and cyclically moving visual object. In some embodiments, non-predictable stimuli are presented to a subject before the predictable stimuli. The subject's responses to visual stimuli are typically visual, and in some of such embodiments, the subject's responses are measured by tracking eye movement. In some embodiments, a frontal brain electroencephalographic (EEG) signal (e.g., the “contingent negative variation” signal) is measured during the period in which a subject responds to the stimuli presented to the subject. The amplitude of the EEG signal is proportional to the degree of anticipation and will be disrupted when there are anticipatory timing deficits.

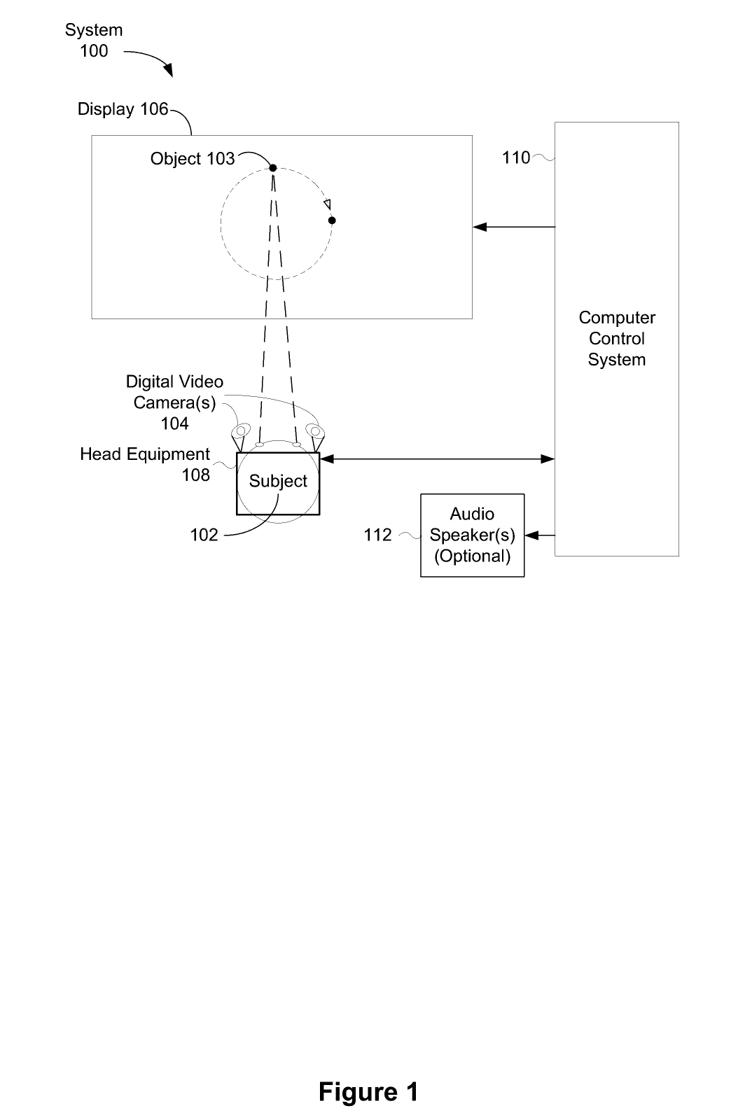

FIG. 1 illustrates a system 100 for measuring a subject's ability to visually track a moving object having predictable movements, typically a repeatedly performed sequence of movement, in accordance with some embodiments. More specifically, system 100 is configured to measure a subject's ability to visually track a smoothly moving object, in accordance with some embodiments. In some embodiments, the smoothly moving object is an object that moves along a continuous path (e.g., a circular path, or oval or elliptical path, rectangular path, or other continuous path) with a rate of movement that is constant, or a rate of movement that is the same at each location along the path each time the object moves through the path, or a rate of movement that follows a regular pattern discernable by ordinary human observers. However, in some other embodiments, movement of the object is continuous over a portion of the object's path, with a rate of movement that is constant or smoothly varying, and is non-continuous over another portion of the object's path (e.g., the object skips over certain portions of the path). In both types of embodiments, however, movement of the object is predictable by normal subjects due to the object's repeated movement over the same path.

In some embodiments, subject 102 is shown smoothly moving image 103 (e.g., a dot or ball moving at a constant speed), following a path (e.g., a circular or oval path) on display 106 (e.g., a screen). Measurement apparatus, such as digital video cameras 104, are focused on subject 102's eyes so that eye positions (and, in some embodiments, eye movements) of subject 102 are recorded. In accordance with some embodiments, digital video cameras 104 are mounted on subject 102's head by head equipment 108 (e.g., a headband or headset). Various mechanisms are, optionally, used to stabilize subject 102's head, for instance to keep the distance between subject 102 and display 106 fixed, and to also keep the orientation of subject 102's head fixed as well. In one embodiment, the distance between subject 102 and display 106 is kept fixed at approximately 40 cm. In some implementations, head equipment 108 includes the head equipment and apparatuses described in U.S. Patent Publication 2010/0204628 A1, which is incorporated by reference in its entirety. In some embodiments, the display 106, digital video cameras 104, and head equipment 108 are incorporated into a portable headset, configured to be worn by the subject while the subject's ability to track the smoothly moving object is measured. In some embodiments, head equipment 108 includes the headset described in U.S. Pat. No. 9,004,687, which is incorporated by reference in its entirety.

Display 106 is, optionally, a computer monitor, projector screen, or other display device. Display 106 and digital video cameras 104 are coupled to computer control system 110. In some embodiments, computer control system 110 controls the display of object 103 and any other patterns or objects or information displayed on display 106, and also receives and analyses the eye position information received from the digital video cameras 104.

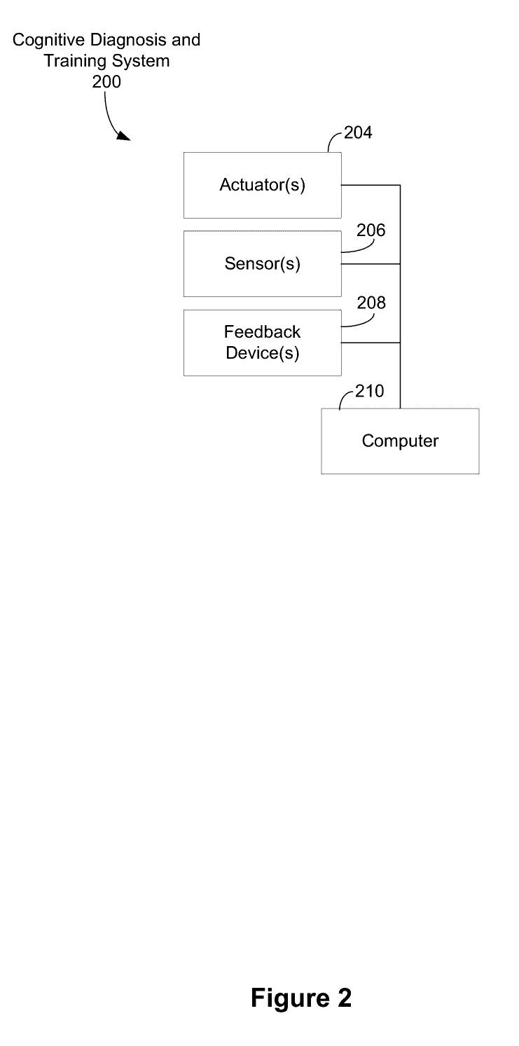

FIG. 2 illustrates a conceptual block diagram of a cognition diagnosis system 100, or a cognition and training system 200, in accordance with some embodiments. System 200 includes computer 210 (e.g., computer control system 110, FIG. 1) coupled to one or more actuators 204, and one or more sensors 206. In some embodiments, system 200 includes one or more feedback devices 208 (e.g., when system 200 is configured for use as a cognitive timing training system). In some embodiments, feedback is provided to the subject via the actuators 204. In some embodiments, actuators 204 include a display device (e.g., display 106, FIG. 1) for presenting visual stimuli to a subject. More generally, in some embodiments, actuators 204 include one or more of the following: a display device for presenting visual stimuli to a subject, audio speakers (e.g., audio speakers 112, FIG. 1) for presenting audio stimuli, a combination of the aforementioned, or one or more other devices for producing or presenting sequences of stimuli to a subject. In some embodiments, sensors 206, are, optionally, mechanical, electrical, electromechanical, auditory (e.g., microphone), or visual sensors (e.g., a digital video camera), or other type of sensors (e.g., a frontal brain electroencephalograph, sometimes called an EEG). The primary purpose of sensors 206 is to detect responses by a subject (e.g., subject 102 in FIG. 1) to sequences of stimuli presented by actuators 204. Some types of sensors produce large amounts of raw data, only a small portion of which can be considered to be indicative of the user response. In such systems, computer 210 contains appropriate filters and/or software procedures for analyzing the raw data so as to extract “sensor signals” indicative of the subject's response to the stimuli. In embodiments in which sensors 206 include an electroencephalograph (EEG), the relevant sensor signals from the EEG may be a particular component of the signals produced by the EEG, such as the contingent negative variation (CNV) signal or the readiness potential signal.

Feedback devices 208 are, optionally, any device appropriate for providing feedback to the subject (e.g., subject 102 in FIG. 1). In some embodiments, feedback devices 208 provide real time performance information to the subject corresponding to measurement results, which enables the subject to try to improve his/her anticipatory timing performance. In some embodiments, the performance information provides positive feedback to the subject when the subject's responses (e.g., to sequences of stimuli) are within a normal range of values. In some embodiments, the one or more feedback devices 208 may activate the one or more actuators 204 in response to positive performance from the subject, such as by changing the color of the visual stimuli or changing the pitch or other characteristics of the audio stimuli.

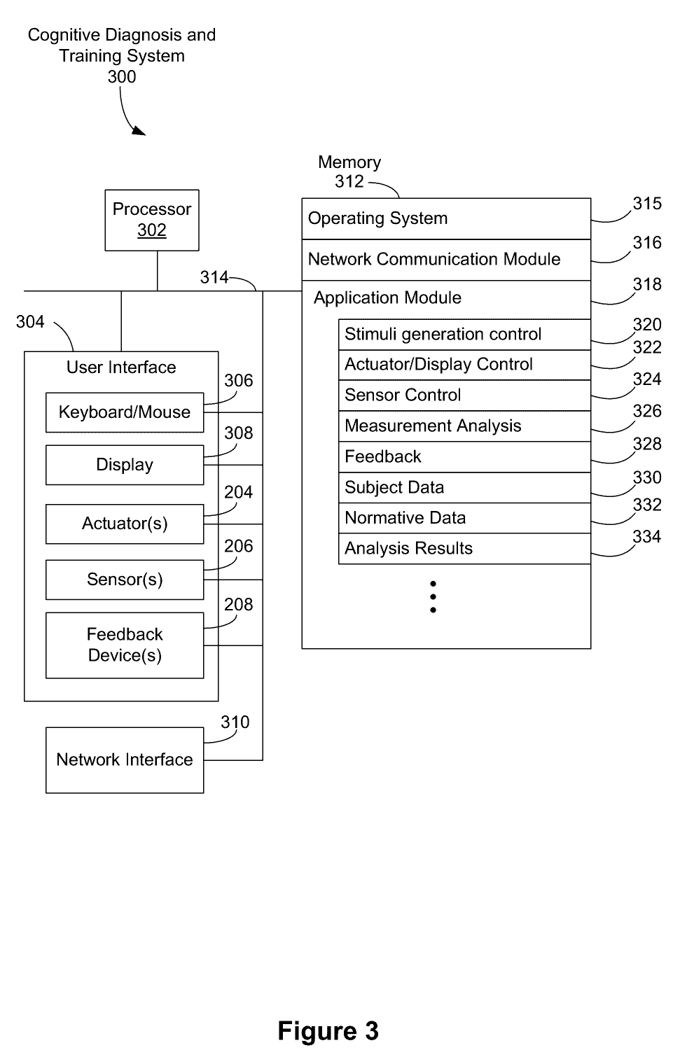

FIG. 3 is a block diagram of a cognition timing diagnosis and training (or remediation) system 300 in accordance with some embodiments. System 300 includes one or more processors 302 (e.g., CPUs), user interface 304, memory 312, and one or more communication buses 314 for interconnecting these components. In some embodiments, system 300 includes one or more network or other communications interfaces 310, such as a network interface for conveying testing or training results to another system or device. User interface 304 includes at least one or more actuators 204 and one or more sensors 206, and, in some embodiments, also includes one or more feedback devices 208. In some embodiments, actuator(s) 204 and sensor(s) 206 are implemented in a headset, while the remaining elements are implemented in a computer system coupled (e.g., by a wired or wireless connection) to the headset. In some embodiments, the user interface 304 includes computer interface devices such as keyboard/mouse 306 and display 308.

In some implementations, memory 312 includes a non-transitory computer readable medium, such as high-speed random access memory and/or non-volatile memory (e.g., one or more magnetic disk storage devices, one or more flash memory devices, one or more optical storage devices, and/or other non-volatile solid-state memory devices). In some implementations, memory 312 includes mass storage that is remotely located from processing unit(s) 302. In some embodiments, memory 312 stores an operating system 315 (e.g., Microsoft Windows, Linux or Unix), an application module 318, and network communication module 316.

In some embodiments, application module 318 includes stimuli generation control module 320, actuator/display control module 322, sensor control module 324, measurement analysis module 326, and, optionally, feedback module 328. Stimuli generation control module 320 generates sequences of stimuli, as described elsewhere in this document. Actuator/display control module 322 produces or presents the sequences of stimuli to a subject. Sensor control module 324 receives sensor signals and, where appropriate, analyzes raw data in the sensor signals so as to extract sensor signals indicative of the subject's (e.g., subject 102 in FIG. 1) response to the stimuli. In some embodiments, sensor control module 324 includes instructions for controlling operation of sensors 206. Measurement analysis module 326 analyzes the sensor signals to produce measurements and analyses, as discussed elsewhere in this document. Feedback module 328, if included, generates feedback signals for presentation to the subject via the one or more actuators or feedback devices.

In some embodiments, application module 318 furthermore stores subject data 330, which includes the measurement data for a subject, and analysis results 334 and the like. In some embodiments, application module 318 stores normative data 332, which includes measurement data from one or more control groups of subjects, and optionally includes analysis results 334, and the like, based on the measurement data from the one or more control groups.

Still referring to FIG. 3, in some embodiments, sensors 206 include one or more digital video cameras focused on the subject's pupil (e.g., digital video cameras 104), operating at a picture update rate of 30 hertz or more. In some embodiments, the one or more digital video cameras are infrared cameras, while in other embodiments, the cameras operate in other portions of the electromagnetic spectrum. In some embodiments, the resulting video signal is analyzed by processor 302, under the control of measurement analysis module 326, to determine the screen position(s), sometimes herein called gaze positions, where the subject focused, and the timing of when the subject focused at one or more predefined screen positions. For purposes of this discussion, the location of a subject's focus is the center of the subject's visual field. For example, using a picture update rate of 100 hertz, during a predefined test period of N seconds (e.g., 30 seconds), N×100 gaze position measurements are obtained, or 3000 gaze position measurements in 30 seconds. In another example, using a picture update rate of 500 hertz, during a predefined test period of N seconds (e.g., 30 seconds), N×500 gaze position measurements are obtained, or 15,000 gaze position measurements in 30 seconds.

In some embodiments, not shown, the system shown in FIG. 3 is divided into two systems, one which tests a subject and collects data, and another which receives the collected data, analyzes the data and generates one or more corresponding reports.

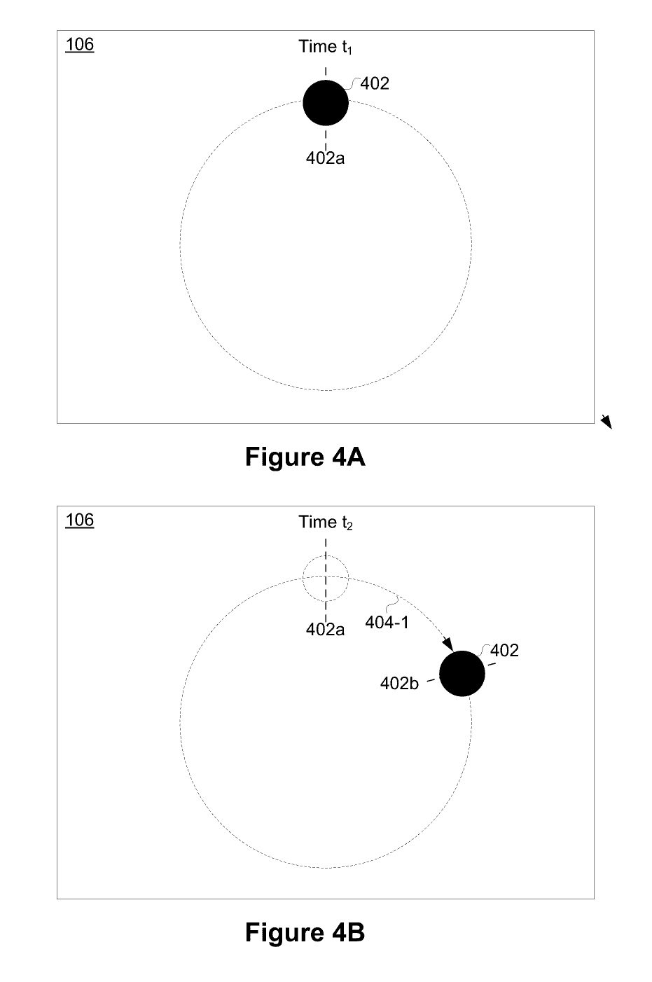

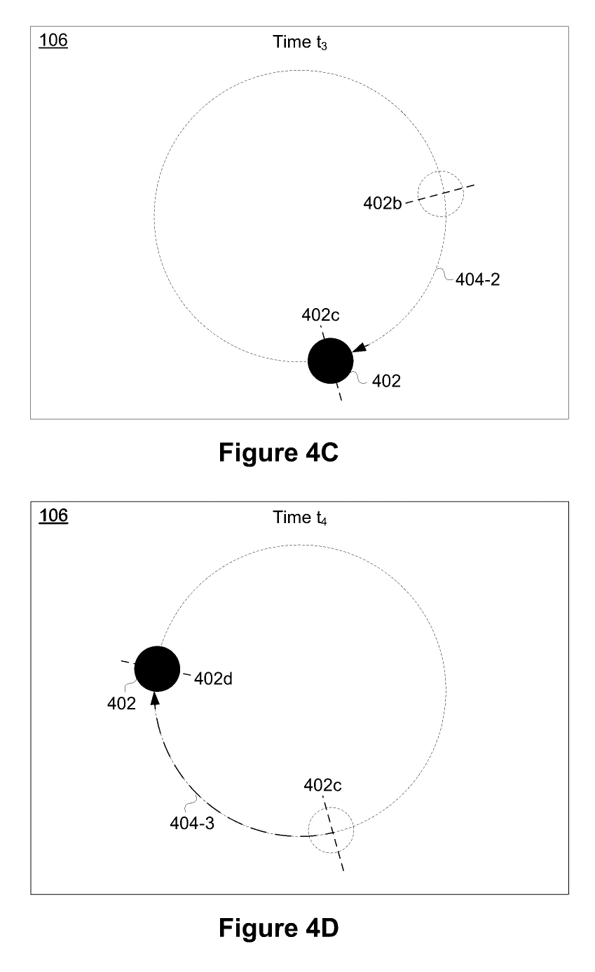

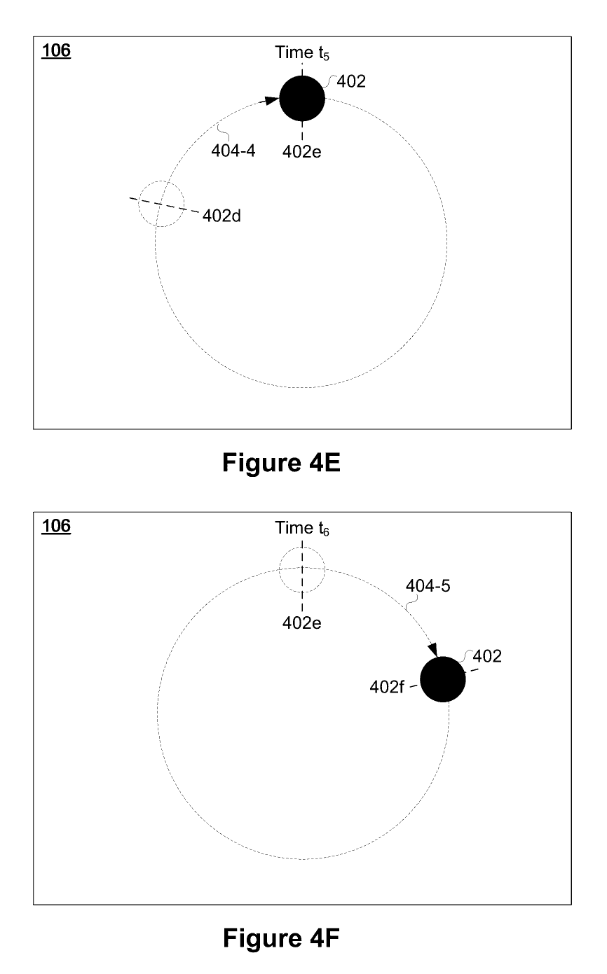

Ocular Pursuit. FIGS. 4A-4F illustrate a smoothly moving object, moving over a tracking path in accordance with some embodiments. FIG. 4A shows object 402 (e.g., a dot) at position 402a on display 106 (on the tracking path) at time t₁. FIG. 4B shows object 402 move along tracking path segment 404-1 to position 402b at time t₂. FIG. 4C shows object 402 move along tracking path segment 404-2 to position 402c at time t₃. FIG. 4D shows object 402 move along tracking path segment 404-3 to position 402d at time t₄. Tracking path segment 404-3 is shown as a dotted line to indicate that object 402 may or may not be displayed while moving from position 402c to position 402d (e.g., tracking path segment 404-3 represents a gap in tracking path 404 of object 402 when object 402 is not displayed on this path segment). FIG. 4E shows object 402 move along tracking path segment 404-4 to position 402e at time t₅. In some embodiments, position 402e is the same as position 402a and time t₅ represents the time it takes object 402 to complete one revolution (or orbit) along the tracking path. FIG. 4F shows object 402 moving along tracking path segment 404-5 to position 402f at time t₆. In some embodiments, position 402f is position 402b.

For purposes of this discussion the terms “normal subject” and “abnormal subject” are defined as follows. Normal subjects are healthy individuals without any known or reported impairments to brain function. Abnormal subjects are individuals suffering from impaired brain function with respect to sensory-motor or anticipatory timing.

In some embodiments, the width of a subject's anticipatory timing distribution is defined as the variance of the response distribution, the standard deviation of the response distribution, the average deviation of the response distribution, the coefficient of variation of the response distribution, or any other appropriate measurement, sometimes called a statistical measurement, of the width of the response distribution.

The subject's anticipatory timing distribution can be compared with the anticipatory timing distribution of a control group of subjects. Both the average timing and the width of the timing distribution, as well as their comparison with the same parameters for a control group are indicative of whether the subject is suffering from a cognitive timing impairment.

Calibration. In some embodiments, in order to provide accurate and meaningful real time measurements of where the user's is looking at any one point in time, the eye position measurements (e.g., produced via digital video cameras 104) are calibrated by having the subject focus on a number of points on a display (e.g., display 106) during a calibration phase or process. For instance, in some embodiments, calibration may be based on nine points displayed on the display, include a center point, positioned at the center of the display locations to be used during testing of the subject, and eight points along the periphery of the display region to be used during testing of the subject. The subject is asked to focus on each of the calibration points, in sequence, while digital video cameras (e.g., digital video cameras 104) measure the pupil and/or eye position of the subject. The resulting measurements are then used by a computer control system (e.g., computer control system 110) to produce a mapping of eye position to screen location, so that the system can determine the position of the display at which the user is looking at any point in time. In other embodiments, the number of points used for calibration may be more or less than nine points, and the positions of the calibration points may distributed on the display in various ways.

In some implementations, the calibration process is performed each time a subject is to be tested, because small differences in head position relative to the cameras, and small differences in position relative to the display 106, can have a large impact on the measurements of eye position, which in turn can have a large impact of the “measurement” or determination of the display position at which the subject is looking. The calibration process can also be used to verify that the subject (e.g., subject 102) has a sufficient range of oculomotor movement to perform the test.

Ocular Pursuit to Assess Anticipatory Timing. In some embodiments, after calibration is completed, the subject is told to look at an object (e.g., a dot or ball) on the display and to do his/her best to maintain the object at the center of his/her vision as it moves. In some embodiments, stimuli generation control module 320 generates or controls generation of the moving object and determination of its tracking path, and actuator/display control module 322 produces or presents the sequences of stimuli to the subject. The displayed object is then smoothly moved over a path (e.g., a circular or elliptical path). In some embodiments, the rate of movement of the displayed object is constant for multiple orbits around the path. In various embodiments, the rate of movement of the displayed object, measured in terms of revolutions per second (i.e., Hertz), is as low as 0.1 Hz and as high as 10 Hz. However, it has been found that the most useful measurements are obtained when the rate of movement of the displayed object is in the range of about 0.4 Hz to 1.0 Hz, and more generally when the rate of movement of the displayed object is in the range of about 0.2 Hz to 2.0 Hz. A rate of 0.4 Hz corresponds to 2.5 seconds for the displayed object to traverse the tracking path, while a rate of 1.0 Hz corresponds to 1.0 seconds for the displayed object to traverse the tracking path. Even normal, healthy subjects have been typically found to have trouble following a displayed object that traverses a tracking path at a repetition rate of more than about 2.0 Hz.

In some embodiments, the subject is asked to follow the moving object for eight to twenty clockwise circular orbits. For example, in some embodiments, the subject is asked to follow the moving object for twelve clockwise circular orbits having a rate of movement of 0.4 Hz, measured in terms of revolutions per second. Furthermore, in some embodiments, the subject is asked to follow the moving object for two or three sets of eight to twenty clockwise circular orbits, with a rest period between.

The angular amplitude of the moving object, as measured from the subject's eyes, is about 10 degrees in the horizontal and vertical directions. In other embodiments, the angular amplitude of the moving object, as measured from the subject's eyes, is 15 degrees or more. The eye movement of the subject, while following the moving displayed object, can be divided into horizontal and vertical components for analysis. Thus, in some embodiments, four sets of measurements are made of the subject's eye positions while performing smooth pursuit of a moving object: left eye horizontal position, left eye vertical position, right eye horizontal position, and right eye vertical position. Ideally, in such embodiments as those utilizing a circularly or elliptically moving visual object, if the subject perfectly tracked the moving object at all times, each of the four positions would vary sinusoidally over time. That is, a plot of each component (horizontal or vertical) of each eye's position over time would follow the function sin(ωt+θ), where sin( )) is the sine function, θ is an initial angular position, and ω is the angular velocity of the moving object. In some embodiments, one or two sets of two dimensional measurements (based on the movement of one or two eyes of the subject) are used for analysis of the subject's ability to visually track a smoothly moving displayed object. In some embodiments, the sets of measurements are used to generate a tracking metric. In some embodiments, the sets of measurements are used to generate a disconjugacy metric by using a binocular coordination analysis.

In some embodiments, the subject is asked to focus on an object that is not moving, for a predefined test period of T seconds (e.g., 30 seconds, or any suitable test period having a duration of 15 to 60 seconds), measurements are made of how well the subject is able to maintain focus (e.g., the center of the subject's visual field) on the object during the test period, and an analysis, similar to other analyses described herein, is performed on those measurements. In some circumstances, this “non-moving object” test is performed on the subject in addition to the ocular pursuit test(s) described herein, and results from the analyses of measurements taken during both types of tests are used to evaluate the subjects cognitive function.

Ocular pursuit eye movement is an optimal movement to assess anticipatory timing in intentional attention (interaction) because it requires attention. Measurements of the subject's point of focus, defined here to be the center of the subject's visual field, while attempting to visually track a moving displayed object can be analyzed for binocular coordination so as to generate a disconjugacy metric. Furthermore, as discussed in more detail in published U.S. Patent Publication 2006/0270945 A1, which is incorporated by reference in its entirety, measurements of a subject's point of focus while attempting to visually track a moving displayed object can also be analyzed so as to provide one or more additional metrics, such as a tracking metric, a metric of attention, a metric of accuracy, a metric of variability, and so on.

In accordance with some implementations, for each block of N revolutions or orbits of the displayed object, the pictures taken by the cameras are converted into display locations (hereinafter called subject eye positions), indicating where the subject was looking at each instant in time recorded by the cameras. In some embodiments, the subject eye positions are compared with the actual displayed object positions. In some embodiments, the data representing eye and object movements is low-pass filtered (e.g., at 50 Hz) to reduce signal noise. In some embodiments, saccades, which are fast gaze shifts, are detected and counted. In some embodiments, eye position measurements during saccades are replaced with extrapolated values, computed from eye positions preceding each saccade. In some other embodiments, eye position and velocity data for periods in which saccades are detected are removed from the analysis of the eye position and velocity data. The resulting data is then analyzed to generate one or more of the derived measurements or statistics discussed below.

Disconjugacy of Binocular Coordination. Many people have one dominant eye (e.g., the right eye) and one non-dominant eye (e.g., the left eye). For these people, the non-dominant eye follows the dominant eye as the dominant eye tracks an object (e.g., object 103 in FIG. 1, or object 402 in FIGS. 4A-4F). In some embodiments, a disconjugacy metric is calculated to measure how much the non-dominant eye lags behind the dominant eye while the dominant eye is tracking an object. Impairment due to sleep deprivation, aging, alcohol, drugs, hypoxia, infection, clinical neurological conditions (e.g., ADHD, schizophrenia, and autism), and/or brain trauma (e.g., head injury or concussion) can increase the lag (e.g., in position or time) or differential (e.g., in position or time) between dominant eye movements and non-dominant eye movements, and/or increase the variability of the lag or differential, and thereby increase the corresponding disconjugacy metric.

In some embodiments, the disconjugacy of binocular coordination is the difference between the left eye position and the right eye position at a given time, and is calculated as:

Disconj(t)=POS_{LE}(t)−POS_{RE}(t)

where “t” is the time and “POS_{LE}(t)” is the position of the subject's left eye at time t and “POS_{RE}(t)” is the position of the subject's right eye at time t. In various embodiments, the disconjugacy measurements include one or more of: the difference between the left eye position and the right eye position in the vertical direction (e.g., POS_{RE}_{ₓ}(t) and POS_{LE}_{ₓ}(t)); the difference between the left eye position and the right eye position in the horizontal direction (e.g., POS_{RE}_{_{y}}(t) and POS_{LE}_{_{y}}(t)); the difference between the left eye position and the right eye position in the two-dimensional horizontal-vertical plane (e.g., POS_{RE}_{_{xy}}(t) and POS_{LE}_{_{xy}}(t)); and a combination of the aforementioned.

In some embodiments, a test includes three identical trials of 12 orbits. To quantify the dynamic change of disconjugacy during a test, the data from each trial is aligned in time within each test and the standard deviation of disconjugate eye positions (SDDisconj) is calculated. In accordance with some embodiments, SDDisconj for a set of “N” values is calculated as:

$${SDDisconj}_{N} = \sqrt{\frac{1}{N} {\sum }_{i = 1}^{N} {( {x}_{i} - 〈 x 〉 )}^{2}}$$

where “x” is a disconjugate measurement discusssed above (e.g., Disconj(t)) and “(x)” represents the average value of the disconjugate eye positions. Thus, in various embodiments, SDDisconj_{N} represents: the standard deviation of disconjugate eye positions in the vertical direction; the standard deviation of disconjugate eye positions in the horizontal direction; or the standard deviation of disconjugate eye positions in the two-dimensional horizontal-vertical plane. In some embodiments, a separate SDDisconj measurement is calculated for two or more of the vertical direction, the horizontal direction, and the two-dimensional horizontal-vertical plane.

Therefore, in various embodiments, disconjugacy measurements, standard deviation of disconjugacy measurements, tracking measurements, and related measurements (e.g., a variability of eye position error measurement, a variability of eye velocity gain measurement, an eye position error measurement, a rate or number of saccades measurement, and a visual feedback delay measurement) are calculated. Furthermore, in various embodiments, the disconjugacy measurements, standard deviation of disconjugacy measurements, tracking measurements, and related measurements are calculated for one or more of: the vertical direction; the horizontal direction; the two-dimensional horizontal-vertical plane; and a combination of the aforementioned.

In some embodiments, one or more of the above identified measurements are obtained for a subject and then compared with the derived measurements for other individuals. In some embodiments, one or more of the above identified measurements are obtained for a subject and then compared with the derived measurements for the same subject at an earlier time. For example, changes in one or more derived measurements for a particular person are used to evaluate improvements or deterioration in the person's ability to anticipate events. Distraction and fatigue are often responsible for deterioration in the person's ability to anticipate events and can be measured with smooth pursuit eye movements. In some embodiments, decreased attention, caused by fatigue or a distractor, can be measured by comparing changes in one or more derived measurements for a particular person. In some embodiments, decreased attention can be measured by monitoring error and variability during smooth eye pursuit.

Anticipatory Saccades As Evidence of Neurological Abnormality. Analysis of the results produced by testing of traumatic brain injury patients using the smooth pursuit methodology described herein shows that patients of concussive head injury show deficits in synchronizing their gaze with the target motion during circular visual tracking, while still engaged in predictive behavior per se. The deficits have been characterized with the presence of saccades that carry the gaze a great distance ahead of target relative to those typically observed in normal individuals. Since the destinations of these saccades follow the circular path of the target, the saccades are anticipatory and are therefore herein called anticipatory saccades.

As described in more detail below, characterizing the frequency and amplitudes of anticipatory saccades, as well as overall gaze position error variability in concussed patients, has been found to provide useful indications of functional damage from the injury, and also for measuring or tracking recovery from the injury.

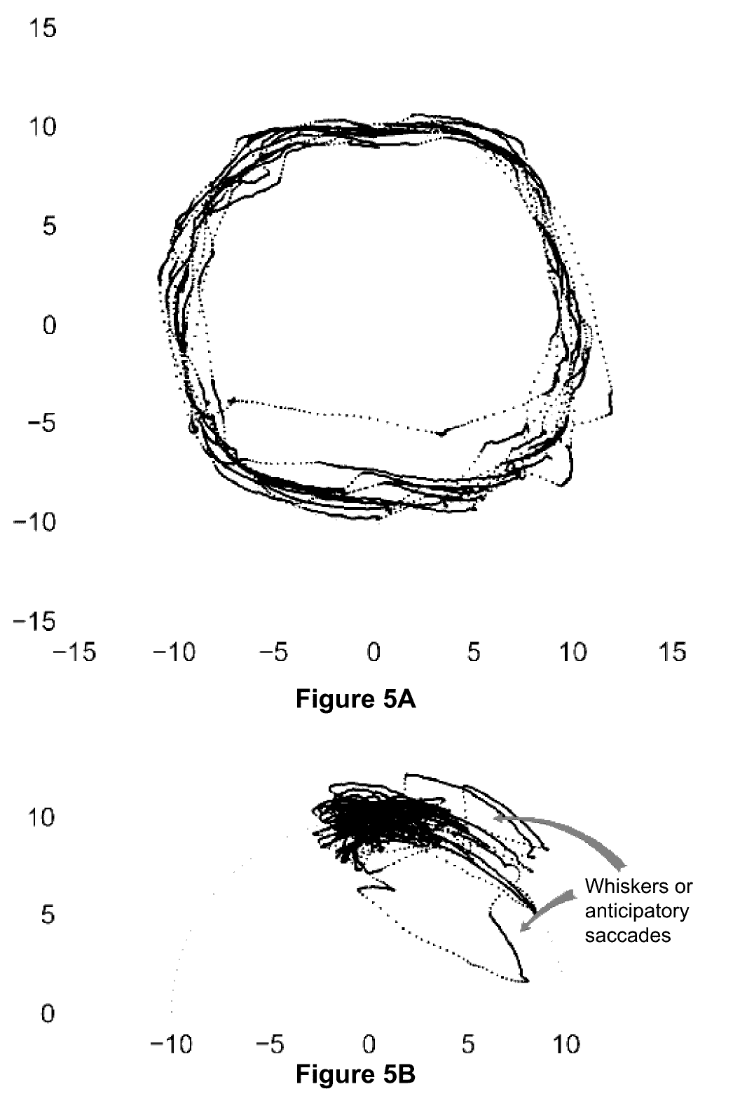

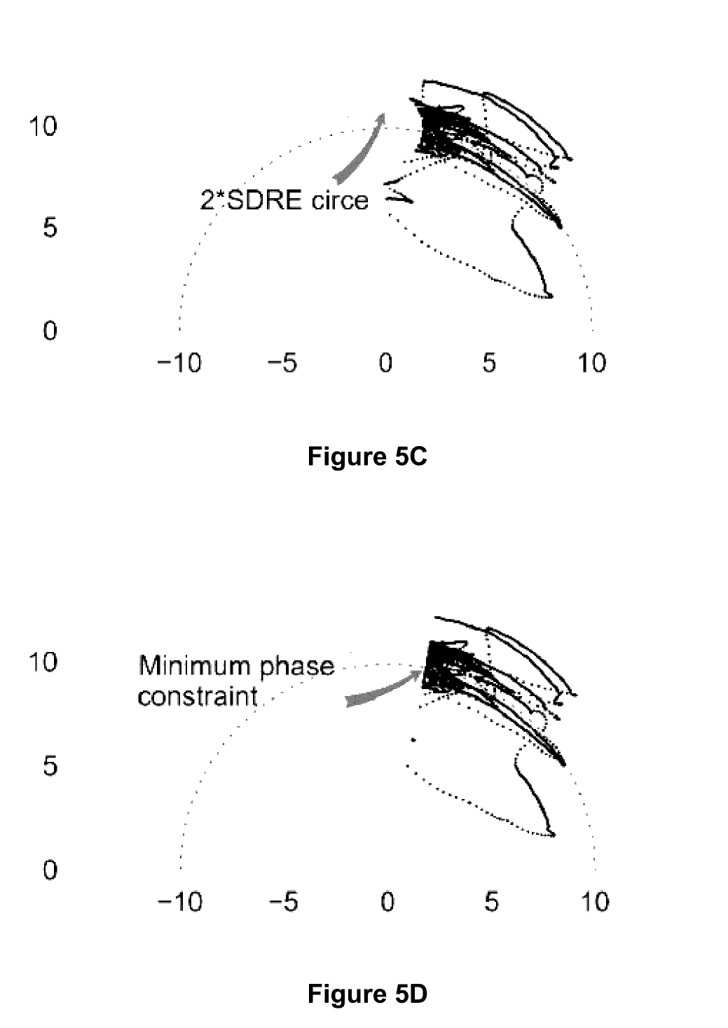

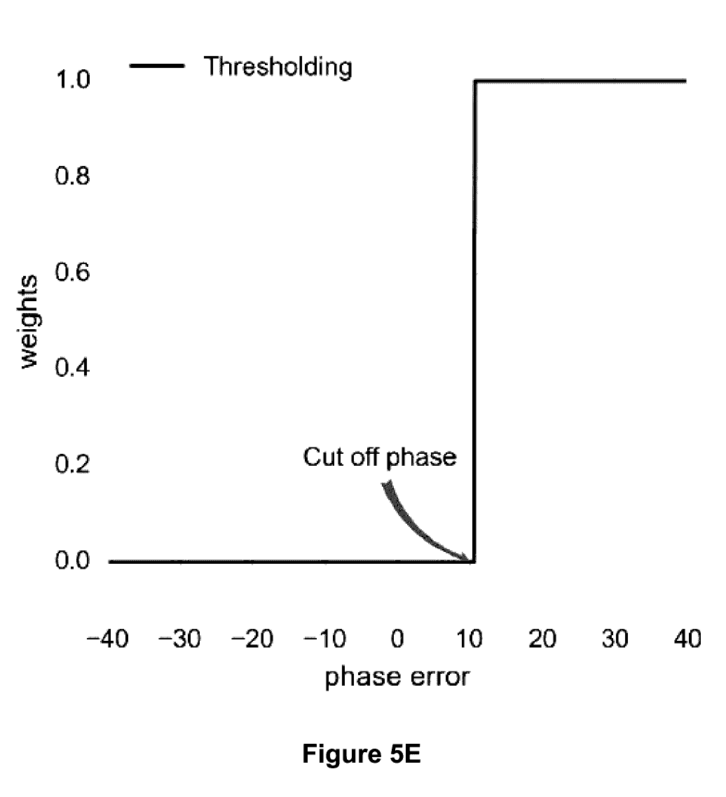

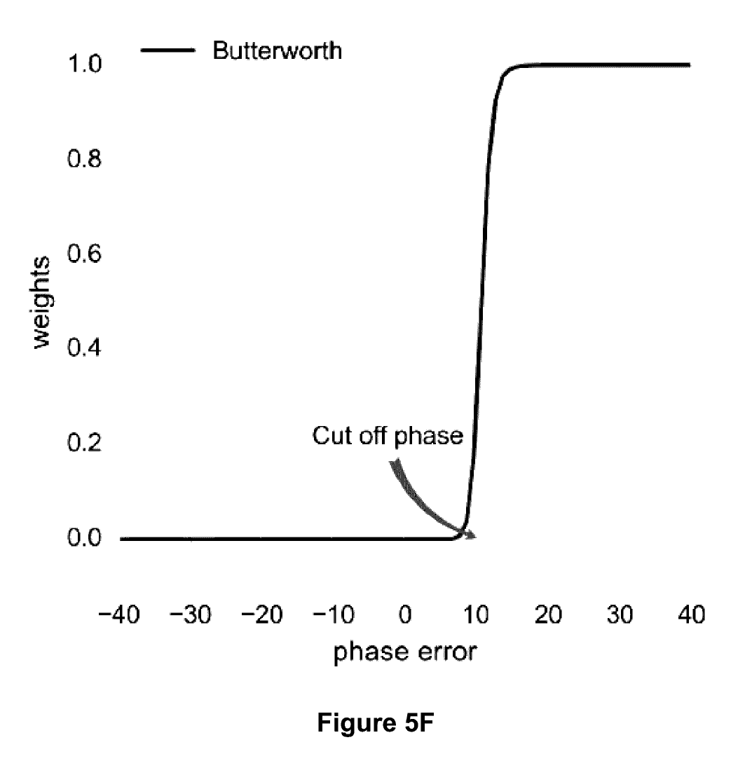

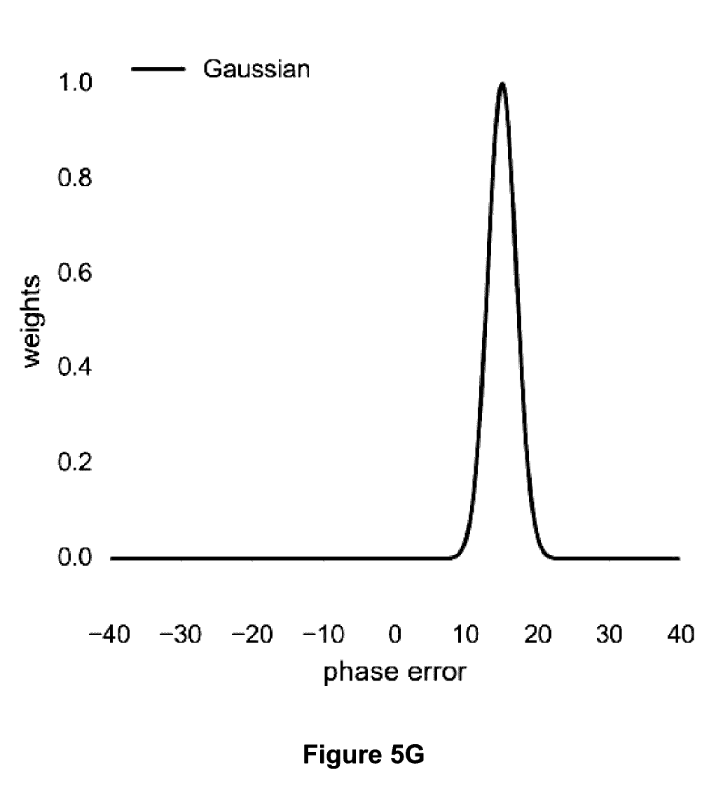

FIG. 5A shows typical eye movements of a subject having concussive head injury, following a target moving along a circular path with a 10 degree radius in visual angle. FIG. 5B shows the same eye movements, plotted in a target-based reference frame in which the target, actually moving clockwise, is fixed at the 12 o'clock position. A gaze point plotted at the 12 o'clock position on the circular path of the target is said to have a zero error. Thus, the data points shown in FIG. 5B represent the subject's tracking errors over the course of the test period, with the tracking errors being shown in two dimensions, radial and tangential, relative to the circular trajectory of the target. FIG. 5B shows the trajectories of anticipatory saccades as arcs or traces that extend in the clockwise direction from the zero error position. These traces are sometimes herein called “whiskers,” to distinguish them from other traces that fall within a predefined zone or range near the zero error position.

The error in the position between the subject's gaze position and the target position at a given time instant can be decomposed into radial and tangential components defined relative to the target trajectory. The radial component represents the subject's spatial error in a direction orthogonal to the target trajectory, whereas the tangential component represents a combination of spatial and temporal errors in a direction parallel to the target trajectory.

While the tracking errors can be characterized as having horizontal (x) and vertical (y) components, it has been found to be useful to characterize the tracking errors as having a radial component and tangential component, for purposes of analyzing the tracking errors and generating metrics concerning the frequency and amplitude of anticipatory saccades.

Next described are methods for detecting anticipatory saccades, which are eye movements that place the subject's gaze further ahead of the target than expected from the subject's general spatial control ability. To aid detection, we note that whiskers have two main characteristics: 1) the whiskers are deviations from the predictable performance of the subject in controlling the subject's gaze position, as determined by statistical analysis of tracking error data produced while the subject visually tracks a smoothly moving object on a display; and 2) the whiskers of interest are always ‘ahead’ of the target's position, and thus have a positive phase with respect to the target's position.

In some embodiments, a region (in the two-dimensional plot of tracking errors) around the zero error position, corresponding to a predictable range of tracking errors, is determined for a subject. Tracking errors falling within this region (e.g., tracking errors having a magnitude less than a determined threshold) are associated with normal spatial control ability of typical, healthy subjects, which includes a certain amount of natural variability and optionally includes a normal level of reduced control ability due to fatigue. But tracking errors falling outside this region are indicative of a loss of anticipatory timing control ability due to concussion or other neurological conditions.

In some embodiments, a statistical measurement, such as the standard deviation of radial errors, SDRE, is determined (as described in more detail below) and used as an estimate of the subject's predictable spatial tracking error. Assuming that the spatial errors are isotropic (i.e., the same in all directions), we define a circular region of radius=2×SDRE around the zero-error position to represent the range of predictable gaze errors for a subject. Gaze position errors that lie outside this region, and that have a positive phase with respect to the target, characterize reduced temporal accuracy or precision in the subject's visual tracking. In some embodiments, gaze position errors that have negative phase are also excluded from the tracking error data that is used to identify and characterize anticipatory saccades. It is noted that the radius of the 2×SDRE circular region is not fixed. In particular, the radius adapts to the subject's performance.

Thus, in some embodiments, tracking error data (produced while a subject visually tracks a moving object on a display during a predefined test period) is filtered to remove data meeting one or more predefined thresholds (e.g., a phase threshold, to exclude tracking errors having negative phase, and an amplitude threshold to exclude tracking errors having amplitude or magnitude less than 2×SDRE) so as to generate filtered tracking error data, an example of which is shown in FIG. 5C. Stated another way, FIG. 5C depicts a plot of a subject's tracking errors over the course of a test period, excluding tracking errors having negative phase and tracking errors that fall within the defined region.

FIG. 5D depicts another example of filtered tracking error data, in which tracking error data, produced while a subject visually tracks a smoothly moving object on a display during a predefined test period, is filtered to remove data having a phase error less than a threshold corresponding to an error of 2×SDRE in the positive tangential direction, so as to generate the filtered tracking error data shown in FIG. 5D.

Quantifying Anticipatory Saccades. As described above, anticipatory saccades are a consequence of saccadic eye movements that result in shifting of the gaze ahead of the target. In some embodiments, anticipatory saccades can be identified as saccades that satisfy a velocity or acceleration threshold, with the added constraint that the phase of the saccades be larger than a minimum phase constraint (e.g., discussed above with respect to FIG. 5D). The number of such anticipatory saccades over the course of a predefined test period, or the frequency of such anticipatory saccades per unit of time, can then be used as one measure of a subject's cognitive impairment or one measure of a subject's concussive injury.

Another metric of a subject's cognitive impairment or concussive injury is a metric of the sizes of the subject's anticipatory saccades during circular visual tracking, quantified as distances, in visual angle for example, covered by the anticipatory saccades, or by a phase-related metric derived from end points of the anticipatory saccades.

Yet another metric of a subject's cognitive impairment or concussive injury is a metric of variability of the filtered tracking error data for the subject's anticipatory saccades during circular visual tracking, for example a standard deviation of tangential errors (also herein called phase errors) associated with anticipatory saccades, excluding tracking error data points having a tangential error (phase error) less than a predefined threshold. Furthermore, as discussed below, phase constraints on what tracking error data to include in the determination of each metric can be handled by applying a weighting function to the tracking error data.

In the mathematical expressions provided below with respect to a subject's gaze position and the position of a target, the variable i represents a time, sometimes called a time instant; xₑ[i] represents horizontal position of the subject's gaze position (in degrees of visual angle) at time instant i; and yₑ[i] represents vertical position of the subject's gaze position (in degrees of visual angle) at time instant i.

In some embodiments, during a predefined test period, the object being displayed on a display screen or device for visual tracking moves along a circular path having a radius R around a center position represented by (0,0). The radial distance of the subject's gaze position from the center of the screen is denoted by rₑ[i]=\sqrt{xₑ²[i]+yₑ²[i]}. The instantaneous radial error between the subject's gaze position and the target at each time instant, i, is given by rₑᵣᵣ[i]=rₑ[i]−R. Furthermore, we define the instantaneous phase error of the gaze position with respect to the target at each time instant, i, to be φₑᵣᵣ[i]. In other embodiments, R[i] may be defined in terms of the instantaneous curvature of the target trajectory and rₑᵣᵣ[i] as the distance between the instantaneous gaze position and the origin that defines the instantaneous curvature of the target trajectory.

Given these representations of the target and the gaze positions, the standard deviation of the radial error (SDRE) is computed based on the radial error at each time point during the predefined test period, as follows:

SDRE(rₑᵣᵣ)=\sqrt{Σᵢ₌₁^{N}(rₑᵣᵣ[i]−r̅ₑ̅ᵣ̅ᵣ̅)²/(N−1)},

where

$$\overline{{r}_{err}} = \frac{1}{N} {\sum }_{i = 1}^{N} ( {r}_{err} [ i ] ) ,$$

is the mean radial error and N represents the total number of data points (i.e., the number of gaze positions measured during the predefined test period).

In some embodiments, a statistical measurement comprising the standard deviation of the tangential error (SDTE) is defined as the standard deviation of the tangential error (phase error) projected along the average gaze trajectory and expressed in units of the degrees of visual angle, and is computed as follows:

$$SDTE ( {φ}_{err} , {r}_{e} ) = \overline{{r}_{e}} {\sum }_{i = 1}^{N} {( {φ}_{err} [ i ] - \overline{{φ}_{err}} )}^{2} / ( N - 1 )$$

$$where , \\ \overline{{φ}_{err}} = \frac{1}{N} {\sum }_{n = 1}^{N} ( {φ}_{err} [ i ] ) ,$$

is the mean phase error, and

$$\overline{{r}_{e}} = \frac{1}{N} {\sum }_{i = 1}^{N} ( {r}_{e} [ i ] ) ,$$

is the mean radial position of the subject's gaze position.

In some embodiments, the threshold error magnitude, S, is determined as follows, where R is the radius of the target circle (i.e., the circular path along which an object is displayed on a display screen or device for visual tracking by the subject) and S is the radius of the circle that defines a 2*SDRE circular region around the target. The minimum phase angle can be defined to be

$${φ}_{min} = 2 \times {sin}^{- 1} ( \frac{S}{2 R} ) .$$

In some embodiments, when filtering the tracking error data to produce filtered tracking error data, tracking errors having a phase less than the minimum phase angle, φₘᵢₙ, are filtered out, or given zero weight using a weighting function shown in FIG. 5E. One way to implement such a weighting function is as follows. For a particular value of phase error, φₑᵣᵣ, and the minimum phase angle, φₘᵢₙ, a first weighting function w[i] is defined as

$$w [ i ] = w ( {φ}_{err} [ i ] ) = \begin{matrix}0 if {φ}_{err} [ i ] \leq {φ}_{min} \\ 1 if {φ}_{err} [ i ] > {φ}_{min}\end{matrix}$$

In other words, this weighting function (which can also be called a threshold function since it gives zero weight to tracking errors that do not satisfy a threshold), retains only the phase errors whose values are greater than φₘᵢₙ.

In some embodiments, given the phase error of a subject's gaze position with respect to the target's position, φₑᵣᵣ[i], the mean radius of the subject's gaze position, r̅ₑ̅, and a weighting function, w[i], such as the example discussed above, a metric for quantifying anticipatory saccades is computed as a weighted standard deviation of the phase error measured in units of degrees of visual angle:

$${SDTE}_{whisk} ( {φ}_{err} , {\overline{r}}_{e} ) = \overline{{r}_{e}} \sqrt{\frac{{\sum }_{i = 1}^{N} w [ i ]}{{( {\sum }_{i = 1}^{N} w [ i ] )}^{2} - {\sum }_{i = 1}^{N} ( {w}^{2} [ i ] )} {\sum }_{i = 1}^{N} w [ i ] {( {φ}_{err}^{2} [ i ] - {\overline{{φ}_{err}}}^{*} )}^{2}} , \\ where$$

$${\overline{{φ}_{err}}}^{*} = \frac{{\sum }_{i = 1}^{N} w [ i ] {φ}_{err} [ i ]}{{\sum }_{i = 1}^{N} w [ i ]} ,$$

is the weighted mean of the phase error.

In some embodiments, the weighting function applied to the phase errors is not a hard-threshold weighting function, and instead is a weighting function that smoothly transitions between minimum and maximum weights. One example of such a weighting function is a Butterworth-filter-like weighting function, as follows:

$$w [ i ] = w ( {φ}_{err} [ i ] ) = 1 - \frac{1}{1 + {( \frac{{φ}_{err} [ i ]}{{φ}_{min}} )}^{K}}$$

where K is the filter order that controls the rate at which the function's value changes from 0.0 to 1.0. This weighting function, a graphical plot of which is shown in FIG. 5F, takes a value close to 0.0 (e.g., less than 0.05, in the range between 0.05 and 0.0) when φₑᵣᵣ[i]<<φₘᵢₙ thereby discarding gaze positions whose phase error is much smaller than φₘᵢₙ. The weighting function takes a value close to 1.0 (e.g., greater than 0.95, in the range between 0.95 and 1.0) whenφₑᵣᵣ[i]>>φₘᵢₙ, thereby retaining all gaze position whose phase errors are much larger than φₘᵢₙ. However, unlike the thresholding function described earlier, this weighting function has a gradual rise between the two extreme values of 0.0 and 1.0 with a value of 0.5 for φₑᵣᵣ[i]=φₘᵢₙ. It is noted that the smoothing Butterworth-filter-like weighting function gets its cut off parameter from the 2×SDRE circle described above, and therefore is adaptive to the subject's performance.

It is also possible to extend the idea of the smoothing functions to include Gaussian-like windows to select a single (or multiple) ranges of phase errors. FIG. 5G shows an example of a Gaussian weighting that selectively gives a higher weight only to anticipatory saccades whose phase errors are close to 15°.

Testing Methods. In some embodiments, a method of testing a subject for cognitive impairment is performed by a system that includes a computer system, a display, and a measurement apparatus to measure the subject's gaze positions over a period of time while viewing information displayed on the display. The computer system includes one or more processors and memory storing one or more programs for execution by the one or more processors. Under control of the one or more programs executed by the computer system, the method includes, during a predefined test period (e.g., a 30 second period), presenting to a subject, on the display, a moving object, repeatedly moving over a tracking path; and while presenting to the subject the moving object on the display and while the subject visually tracks the moving object on the display, measuring the subject's gaze positions, using the measurement apparatus. For example, as discussed above, the method may include making 100 to 500 measurements of gaze position per second, thereby generating a sequence of 3,000 to 15,000 gaze position measurements over a 30 second test period.

The method further includes, using the computer system (or another computer system to which the measurements or related information is transferred), generating tracking error data corresponding to differences in the measured gaze positions and corresponding positions of the moving object. A visual representation of such tracking error data is shown in FIG. 5B, while FIG. 5A is a visual representation of the measurements.

Next, the method includes filtering the tracking error data to remove or assign weights to data meeting one or more predefined thresholds so as to generate filtered tracking error data. Examples of such filtering are discussed above. In particular, FIG. 5C shows filtered tracking data generated by applying a “circular” filter to the tracking error data, where the circular filter has a radius of two times the standard deviation of the radial error (SDRE), which is computed based on the radial error at each time point during the predefined test period. FIG. 5D shows filtered tracking data generated by filtering out tracking error data having a phase angle less than a minimum phase angle, where the minimum phase angle is determined based on an analysis of the tracking error data, as described in more detail above. Further examples of generating filtered tracking data are provided above through the application of various weighting functions to the tracking error data, and then removing any resulting tracking error data whose resulting value or amplitude is zero.

After filtering the tracking data, the method includes generating one or more metrics based on the filtered tracking error data, the one or more metrics including at least one metric indicative of the presence or absence of anticipatory saccades in subject's visual tracking of the moving object, and then generating a report that includes information corresponding to the one or more metrics. Some examples of such metrics have been discussed above.

In some embodiments, the moving object, presented to the subject on the display, is a smoothly moving object, repeatedly moving over the tracking path.

In some embodiments, the tracking error data includes a sequence of tracking error values, each having a radial error component and a tangential error component (also called a phase error component), and the method includes computing a threshold, of the one or more predefined thresholds, corresponding to a predefined multiple of the standard deviation of the radial error component and/or the phase error component of the tracking error data. Stated somewhat more generally, in some embodiments, the method includes computing a threshold, of the one or more predefined thresholds, corresponding to a predefined multiple of the standard deviation of one or more components of the tracking error data. Examples of such thresholds and how to compute them are provided above.

In some embodiments, the method includes computing a threshold, of the one or more predefined thresholds, corresponding to a predefined multiple of a predefined statistical measurement of one or more components of the tracking error data.

In some embodiments, computing the threshold includes applying a weighting function to the tracking error data to produce weighted tracking error data and computing the predefined statistical measurement with respect to the weighted tracking error data.

In some embodiments, the method further includes generating one or more first comparison results by comparing the one or more metrics with one or more corresponding normative metrics corresponding to performance of other subjects while visually tracking a moving object on a display, and the report is based, at least in part, on the one or more first comparison results. For example, in some embodiments, the one or more metrics for the subject are compared with corresponding metrics for other subjects which known medical conditions or status, and based on those comparisons, a preliminary categorization or evaluation of at least one aspect of the subject's health (e.g., presence, absence or likelihood of concussion, likely severity of concussion, and/or the presence, absence, likelihood, or likely severity of other neurological, psychiatric or behavioral condition) or cognitive performance is included in the report.

In some embodiments, the method further includes generating one or more second comparison results by comparing the one or more metrics with one or more corresponding baseline metrics corresponding to previous performance of the subject while visually tracking a moving object on a display, and the report is based, at least in part, on the one or more second comparison results. For example, soldiers or football players, or any other person may have undergo the testing described herein, while the person is in or appears to be in good health, to generate baseline metrics. Those baseline metrics can then be used as a basis for comparison when the same person is later tested, for example after an accident or other incident that might have caused a concussion or other injury to the person.

In some embodiments, the one or more metrics generated by the method include a metric corresponding to a number or frequency of anticipatory saccades by the subject during the predefined test period. Furthermore, in some embodiments, the one or more metrics include a metric corresponding to an amplitude of anticipatory saccades by the subject during the predefined test period.

In some embodiments, a magnitude of at least one of the one or more metrics corresponds to a degree of impairment of the subject's spatial control.

In some embodiments, the method further includes generating a cognitive impairment metric corresponding to variability of the tracking error data and the report includes information corresponding to the cognitive impairment metric. Examples of such cognitive impairment metrics are taught in U.S. Pat. No. 7,819,818, “Cognition and motor timing diagnosis using smooth eye pursuit analysis,” and U.S. application Ser. No. 14/454,662, filed Aug. 7, 2014, entitled “System and Method for Cognition and Oculomotor Impairment Diagnosis Using Binocular Coordination Analysis,” both of which are hereby incorporated by reference. The combination of one or more such cognitive impairment metrics and one or more metrics based on the filtered tracking error data can provide a doctor or other diagnostician highly useful information in determining the extent and/or nature of a subject's cognitive impairment and the likely cause or causes of such cognitive impairment.

In another aspect, a testing method may include the initial sequence of operations described above, with respect to collecting measurement data while the subject visually tracks a smoothly moving object, generating tracking data and filtering the tracking data. However, in some embodiments, the method includes displaying a visual representation of the filtered tracking error data, the visual representation indicating the frequency and amplitude of anticipatory saccades in subject's visual tracking of the smoothly moving object. In this method, a person (e.g., a doctor or other diagnostician) viewing the visual representation of the filtered tracking error data can visually discern the frequency and amplitude of anticipatory saccades, if any, by the subject during the predefined test period. Furthermore, the person viewing the visual representation of the filtered tracking error data can discern patterns in the visual representation of the filtered tracking error data that correspond to, or are associated with, different classes of medical conditions, different levels of severity of medical conditions, different types of cognitive impairment, different levels of cognitive impairment, and the like.

In some embodiments of the testing methods described herein, measuring the subject's gaze positions is accomplished using one or more video cameras. For example, in some such embodiments, measuring the subject's gaze positions includes measuring the subject's gaze positions at a rate of at least 100 times per second for a period of at least 15 seconds. Further, in some such embodiments, the predefined test period has a duration between 30 second and 120 seconds.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

It will be understood that, although the terms “first,” “second,” etc. may be used herein to describe various elements, these elements should not be limited by these terms. These terms are only used to distinguish one element from another. For example, a first sound detector could be termed a second sound detector, and, similarly, a second sound detector could be termed a first sound detector, without changing the meaning of the description, so long as all occurrences of the “first sound detector” are renamed consistently and all occurrences of the “second sound detector” are renamed consistently. The first sound detector and the second sound detector are both sound detectors, but they are not the same sound detector.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “upon a determination that” or “in response to determining” or “in accordance with a determination” or “upon detecting” or “in response to detecting” that the stated condition precedent is true, depending on the context.

What is claimed is:

1. A method of testing a subject for cognitive impairment, comprising:
at a system having a computer system, a display, and a measurement apparatus to measure the subject's gaze positions over a period of time while viewing information displayed on the display, the computer system having one or more processors and memory storing one or more programs for execution by the one or more processors, performing a set of operations including:
during a predefined test period,
presenting to the subject, on the display, a moving object, repeatedly moving over a tracking path; and
while presenting to the subject the moving object on the display and while the subject visually tracks the moving object on the display, measuring the subject's gaze positions, using the measurement apparatus; and
using the computer system:
generating tracking error data corresponding to differences in the measured gaze positions and corresponding positions of the moving object;
filtering the tracking error data to remove data meeting one or more predefined thresholds so as to generate filtered tracking error data;
generating one or more metrics based on the filtered tracking error data, the one or more metrics including at least one metric indicative of the presence or absence of anticipatory saccades in subject's visual tracking of the moving object; and
generating a report that includes information corresponding to the one or more metrics.

2. The method of claim 1, wherein the moving object, presented to the subject on the display, is a smoothly moving object, repeatedly moving over the tracking path.

3. The method of claim 1, wherein
the tracking error data includes a sequence of tracking error values, each having a radial component and a tangential component in relation to the tracking path; and
the method includes computing a threshold, of the one or more predefined thresholds, corresponding to a predefined multiple of a statistical measurement of the radial component and/or tangential component of the tracking error values in the tracking error data.

4. The method of claim 1, wherein the method includes computing a threshold, of the one or more predefined thresholds, corresponding to a predefined multiple of a predefined statistical measurement of one or more components of the tracking error data.

5. The method of claim 4, wherein computing the threshold includes applying a weighting function to the tracking error data to produce weighted tracking error data and computing the predefined statistical measurement with respect to the weighted tracking error data.

6. The method of claim 1, further comprising:
generating one or more first comparison results by comparing the one or more metrics with one or more corresponding normative metrics corresponding to performance of other subjects while visually tracking a moving object on a display;
wherein the report is based, at least in part, on the one or more first comparison results.

7. The method of claim 1, further comprising:
generating one or more second comparison results by comparing the one or more metrics with one or more corresponding baseline metrics corresponding to previous performance of the subject while visually tracking a moving object on a display;
wherein the report is based, at least in part, on the one or more second comparison results.

8. The method of claim 1, wherein the one or more metrics include a metric corresponding to a number or frequency of anticipatory saccades by the subject during the predefined test period.

9. The method of claim 1, wherein the one or more metrics include a metric corresponding to an amplitude of anticipatory saccades by the subject during the predefined test period.

10. The method of claim 1, wherein a magnitude of at least one of the one or more metrics corresponds to a degree of impairment of the subject's spatial control.

11. The method of claim 1, further including generating a cognitive impairment metric corresponding to variability of the tracking error data; wherein the report includes information corresponding to the cognitive impairment metric.

12. A method of testing a subject for cognitive impairment, comprising:
at a system having a computer system, a display, and a measurement apparatus to measure the subject's gaze positions over a period of time while viewing information displayed on the display, the computer system having one or more processors and memory storing one or more programs for execution by the one or more processors, performing a set of operations including:
during a predefined test period,
presenting to the subject, on the display, a moving object, repeatedly moving over a tracking path; and
while presenting to the subject the moving object on the display and while the subject visually tracks the moving object on the display, measuring the subject's gaze positions, using the measurement apparatus; and
using the computer system:
generating tracking error data corresponding to differences in the measured gaze positions and corresponding positions of the moving object;
filtering the tracking error data to remove data meeting one or more predefined thresholds so as to generate filtered tracking error data; and
displaying a visual representation of the filtered tracking error data, the visual representation indicating frequency and amplitude of anticipatory saccades in subject's visual tracking of the moving object.

13. The method of claim 12, wherein measuring the subject's gaze positions is accomplished using one or more video cameras.

14. The method of claim 13, wherein measuring the subject's gaze positions includes measuring the subject's gaze positions at a rate of at least 100 times per second for a period of at least 15 seconds.

15. The method of claim 12, wherein the predefined test period has a duration between 30 second and 120 seconds.

16. A system of testing a subject for impairment, comprising:
a measurement apparatus to measure the subject's gaze position;
a display;
one or more processors;
memory, the memory storing one or more programs, the one or more programs comprising instructions to:
during a predefined test period, present to the subject, on the display, a moving object, repeatedly moving over a tracking path;
during the predefined test period, while presenting to the subject the moving object on the display and while the subject visually tracks the moving object on the display, measure the subject's gaze positions, using the measurement apparatus; and
generate tracking error data corresponding to differences in the measured gaze positions and corresponding positions of the moving object;
filter the tracking error data to remove data meeting one or more predefined thresholds so as to generate filtered tracking error data;
generate one or more metrics based on the filtered tracking error data, the one or more metrics including at least one metric indicative of the presence or absence of anticipatory saccades in subject's visual tracking of the moving object; and
generate a report that includes information corresponding to the one or more metrics.

17. The system of claim 16, wherein the tracking error data includes a sequence of tracking error values, each having a radial component and a tangential component in relation to the tracking path; and the one or more programs include instructions to compute a threshold, of the one or more predefined thresholds, corresponding to a predefined multiple of a statistical measurement of the radial component or tangential component of the tracking error values in the tracking error data.

18. The system of claim 16, wherein the display and the measurement apparatus comprise an integrated device that includes both the display and the measurement apparatus.

19. The system of claim 16, including a portable headset, configured to be worn by the subject, that includes both the display and the measurement apparatus.

20. A non-transitory computer readable storage medium storing one or more programs, the one or more programs comprising instructions that when executed by one or more processors of a computer system operatively coupled to a display and a measurement apparatus to measure a subject's gaze position, cause a system that includes the computer system, display and measurement apparatus to:
during a predefined test period, present to the subject, on the display, a moving object, repeatedly moving over a tracking path;
during the predefined test period, while presenting to the subject the moving object on the display and while the subject visually tracks the moving object on the display, measure the subject's gaze positions, using the measurement apparatus; and
generate tracking error data corresponding to differences in the measured gaze positions and corresponding positions of the moving object;
filter the tracking error data to remove data meeting one or more predefined thresholds so as to generate filtered tracking error data;
generate one or more metrics based on the filtered tracking error data, the one or more metrics including at least one metric indicative of the presence or absence of anticipatory saccades in subject's visual tracking of the moving object; and
generate a report that includes information corresponding to the one or more metrics.
